(19) United States
(12) Patent Application Publication
(10) Pub. No.: US 2019/0117841 A1
(43) Pub. Date: Apr. 25, 2019

(54) METHOD OF PROCESSING BIOLOGICAL TISSUE

(71) Applicant: St. Jude Medical, Cardiology Division, Inc., St. Paul, MN (US)

(72) Inventor: Paul E. Ashworth, Danbury, WI (US)

(73) Assignee: St. Jude Medical, Cardiology Division, Inc., St. Paul, MN (US)

(21) Appl. No.: 16/096,768

(22) PCT Filed: Apr. 28, 2017
(86) PCT No.: PCT/US2017/030011

§ 371 Date: Oct. 26, 2018

Related U.S. Application Data
(60) Provisional application No. 62/329,279, filed on Apr. 29, 2016.

(51) Int. Cl. A61L 27/36; A01N 1/02

(52) U.S. Cl. CPC A61L 27/3687; A01N 1/0231; A61L 27/3625; A61L 27/3691

(57) ABSTRACT

Methods of preparing biological tissue for bioprosthetic valves include providing fresh biological tissue; treating the tissue with an aqueous cellulose solution for a time sufficient to allow the fluid in the tissue to be replaced by the aqueous cellulose solution; and cross-linking the tissue, wherein the treating step is performed prior to, during, after, or both prior to and after the cross-linking step. The methods may include storing the cellulose-treated and cross-linked tissue in an aqueous solution; or drying the cellulose-treated and cross-linked tissue by a vacuum-drying or lyophilization process, and storing the dried tissue in a dry, ambient environment. The treated tissue may be in the form of a tissue component for a bioprosthetic valve, a valve assembly for a bioprosthetic valve or a fully assembled bioprosthetic valve incorporating the tissue.

### CROSS-REFERENCE TO RELATED APPLICATIONS

The present application claims the benefit of the filing date of U.S. Provisional Patent Application No. 62/329,279 filed Apr. 29, 2016, the disclosure of which is hereby incorporated herein by reference.

### BACKGROUND OF THE INVENTION

Biological tissue is often used in bioprosthetic devices or to repair damaged tissue in a patient. For example, it has become common practice to replace damaged or diseased native heart valves with bioprosthetic valves. The bioprosthetic valve, which is also generally known as a “tissue valve,” is made with tissue of biological origin, such as tissue of porcine or bovine origin. Typically, the biological tissue is chemically cross-linked or fixed with agents such as glutaraldehyde or formaldehyde to prevent rejection when implanted into a recipient, to provide sterilization, and to help stabilize the proteins in the tissue, thereby making the tissue and the bioprosthetic device containing such tissue more durable to withstand prolonged use and millions of cycles of opening and closing under circulatory pressure without fatigue.

It is known that glycosaminoglycans (GAGs) are naturally occurring extracellular matrix compounds that provide inherent hydrophilicity in the tissue. Because of their capability to absorb a large amount of water within the tissue matrix, it is believed that they are very important to the mechanical behavior of the native valve including reducing stress in the tissue and providing lubricity. However, Lovekamp et al., in Biomaterials, 2006 March, 27(8):1507-1518, demonstrate that GAGs are progressively lost during the normal process of tissue fixation. The loss of GAGs may significantly affect the mechanical stability and durability of prosthetic devices incorporating such tissue.

To prevent the transmission of disease-causing microorganisms to the device recipient, the tissue and the bioprosthetic device made therefrom should be sterile. The bioprosthetic device should be stored in a sterile and stable condition from manufacture until use. For example, bioprosthetic heart valves, including surgical and transcatheter heart valves, are typically sterilized and stored in an aldehyde solution (i.e., glutaraldehyde or formaldehyde) prior to use. These solutions help keep the tissue in a hydrated state and kill any microbes that may be attached to the tissue. However, both glutaraldehyde and formaldehyde are irritants and have some inherent level of toxicity. A bioprosthetic device that is stored in such solutions therefore must be extensively rinsed to remove any residual aldehydes prior to implantation.

Attempts have been made to develop a bioprosthetic valve that is substantially free of glutaraldehyde or formaldehyde and in a substantially dry form that is ready for implantation with minimal preparation prior to surgery. One such attempt, disclosed in U.S. Pat. No. 4,300,243, involves a dehydration process in which the tissue is immersed in an organic solvent, such as ethyl alcohol, which removes a substantial amount of water from the tissue components. In U.S. Pat. No. 8,007,992, Tian et al. disclose a method of treating the tissue with a non-aqueous solution comprising a polyhydric alcohol and a C₁₋₃ alcohol and then removing a portion of the treatment solution. However, the tissue components after such dehydration process cannot be rehydrated substantially to their original dimensions.

Chen et al., in U.S. Pat. No. 6,534,004, disclose a process for dry storing bioprosthetic devices comprising a tissue component. The process includes treating the fixed tissue component with an aqueous solution comprising dimensional stabilizers such as polyhydric alcohols or their derivatives. The tissue component is optionally air dried at ambient conditions and then stored in an environment essentially free of liquid for later processing or implantation. Despite the use of a dimensional stabilizer, such process does not provide the tissue with sufficient dimensional stability.

As noted above, the storage of bioprosthetic valves after processing is an important consideration in providing a valve that is suitable for implantation. In that regard, lyophilization, also known as freeze-drying, is an extremely useful technique for tissue storage for surgical applications. However, the lyophilization process typically causes some undesirable effects on the stored tissue. Prior to lyophilization, the tissue may be treated with a lyoprotectant or a cryoprotectant to mitigate or eliminate the formation of ice crystals during the lyophilization process. As disclosed in WO2015031124, such protectants may include a solution containing hydroxyl ethylene starch (HES), sucrose, propylene glycol, or dimethyl sulphoxide (DMSO). However, these lyoprotectants or cryoprotectants may cause changes in the properties of the tissue that may impact the quality of the bioprosthetic valves having tissue treated with these materials.

Therefore, there is a need to develop a method of preparing biological tissue or a bioprosthetic device containing such tissue so as to have good mechanical stability, durability and no undesirable changes or damage to the tissue. Preferably, the prepared tissue is in a dry form.

### BRIEF SUMMARY OF THE INVENTION

The present invention relates to methods of preparing biological tissue or a bioprosthetic device containing such tissue, wherein the tissue is treated with a cellulose solution. The tissue prepared by the methods, or the bioprosthetic device comprising the same, has good mechanical stability and durability. The cellulose treatment can be conducted on tissue prior to, during or after a cross-linking process. Preferably the cellulose treatment can be conducted both prior to and after the cross-linking process. The processed tissue when wet can be sterilized using an appropriate method that is compatible with the wet tissue. Preferably, the process further comprises the step of drying the tissue under vacuum or in a lyophilizer, and the resultant tissue or device made therefrom can be stored in a dry ambient condition. These methods thus provide the advantages of producing tissue or devices with reduced size, volume and weight compared to bioprosthetic tissue or devices stored in a fluid medium; eliminating toxic fluid associated with the tissue or device storage; and reducing changes or damage to the tissue during the drying process. The processed tissue when dried can be sterilized using an appropriate method that is compatible with the dry tissue. Prior to use, the tissue or the device comprising the same can be rinsed or rehydrated, such as by a sterile 0.9 wt % saline solution.

The cellulose, which is non-toxic, includes, but is not limited to, methyl cellulose, ethyl cellulose, hydroxypropyl cellulose and combinations thereof. When tissue is treated with an aqueous cellulose solution, the fluid content of the tissue is replaced with cellulose by passive diffusion, and the tissue can be subjected to cross-linking, vacuum-drying or lyophilization. Without wishing to be bound by any particular theory, the cellulose is a large polymer and potentially could be trapped in the collagen matrix, even after cross-linking. Therefore, it could potentially reduce mechanical shear stress in the tissue and essentially function as a replacement for GAGs. The tissue treated with cellulose shows good mechanical stability and durability. The cellulose treatment may also help prevent change or damage to the tissue properties during the drying process. The residual cellulose in the tissue can be easily removed from the tissue by rinsing or rehydration, such as with a sterile saline solution.

The present invention provides methods of processing biological tissue so as to have good mechanical stability and durability. The tissue is for use in bioprosthetic devices or other heterograft applications. In one embodiment, the bioprosthetic device is a bioprosthetic heart valve.

In one embodiment, the method comprises the steps of fixing fresh biological tissue; and treating the fixed tissue with a cellulose solution for a time sufficient to allow the fluid in the tissue to be replaced by the cellulose solution. In one embodiment, the cellulose-treated fixed tissue is stored in an aqueous solution. The processed tissue when wet can be sterilized using an appropriate sterilization method that is compatible with wet tissue.

Preferably, the method further comprises placing the cellulose-treated fixed tissue under vacuum or in a lyophilizer for drying; and storing the dry tissue in a dry ambient condition. In another embodiment, the processed tissue when dried can be sterilized using an appropriate sterilization method that is compatible with dry tissue.

In a preferred embodiment, prior to the fixing step, the method further comprises treating the fresh biological tissue with a cellulose solution for a time sufficient to allow the fluid in the tissue to be replaced by the cellulose solution. In a variant, the step of treating the fixed tissue with a cellulose solution can be omitted if the fresh biological tissue is first treated with a cellulose solution. In another variant, the step of treating the tissue with a cellulose solution may be performed during the step of fixing the fresh biological tissue.

In one embodiment, the fixing of the fresh biological tissue is performed chemically. When the tissue is treated with a cellulose solution during the fixing of fresh biological tissue, the fresh biological tissue may be treated with a solution comprising cellulose and a cross-linking chemical reagent.

The tissue may be in the form of a tissue component for a bioprosthetic device or other heterograft applications. The device can be a valve assembly for a bioprosthetic heart valve, or a fully assembled bioprosthetic heart valve incorporating the tissue.

The present invention also relates to tissue, a bioprosthetic device or a packaged bioprosthetic device containing the same, wherein the tissue is prepared by the processing described above.

### BRIEF DESCRIPTION OF THE DRAWINGS

These and other features, aspects, and advantages of the present invention will become better understood with regard to the following description, appended claims, and accompanying drawings in which:

FIG. 1 shows an image of a Trifecta® heart valve which was prepared according to the process of Example 2.

FIG. 2 shows an image of a Trifecta® heart valve which was prepared according to the process of Example 3.

### DETAILED DESCRIPTION

Before describing at least one embodiment of the invention in detail, it is to be understood that the invention is not limited in its application to the details set forth in the following description or exemplified by the examples. The invention is capable of other embodiments or of being practiced or carried out in various ways. Also, it is to be understood that the phrasing and terminology employed herein is for the purpose of description and should not be regarded as limiting.

As used herein, the terms “about,” “up to,” “generally,” “substantially” and the like are intended to mean that slight deviations from absolute are included within the scope of the term so modified.

The present invention provides a method of preparing tissue or a bioprosthetic device comprising the same so as to have good mechanical stability and durability. Bioprosthetic valves, which are also generally known as “tissue valves,” are made with tissue of biological origin. “Biological tissue” or “tissue” as used herein refers to biological tissue dissected from an animal such as, for example, porcine or bovine tissue, or tissue from other species. Specific tissue types that may be used include, without limitation, any blood vessel, pericardial tissue, heart muscle tissue, dura mater and the like. More than one species and tissue type may be used in a valve assembly. “Fixed” or “cross-linked” tissue refers to tissue in which the proteins have reduced solubility, antigenicity, and biodegradability as compared to the proteins in the native tissue. “Fixing” or “cross-linking” can be accomplished by a number of techniques, for example, by treatment with aldehydes, epoxides, carbodimides or genipin, or by photo fixation. Conventionally, fixing can be performed by cross-linking the amine groups of the tissue proteins with an aldehyde, such as about 0.001 v/v %-5 v/v % glutaraldehyde or formaldehyde solution. The term “valve” as used herein refers to a complete and operable structure capable of being implanted into a patient to control the flow of blood through the patient's circulatory system. A valve can be a surgical valve, a transcatheter valve or any other non-native valve structure. The terms “implanted” or “implantation” as used herein refer to a complete and long-term seating of a valve in a patient. A “tissue valve” is a bioprosthetic valve that includes at least some tissue of biological origin (also known herein and generally as “bioprosthetic tissue” or “biological tissue”). A “valve assembly” as used herein is a structure that is made, at least in part, from tissue, and that operates to meter or restrict blood flow for at least some period of time, but does not include other structures like a stent often used to support the valve assembly. Thus, a tissue valve for purposes of the present description is a bioprosthetic valve that includes at least a valve assembly. The tissue valve may, and often does, include other structures, such as a supporting stent. Bioprosthetic valves in accordance with the present invention may be used in the heart as a replacement for one of the native cardiac valves, such as the aortic valve or mitral valve. But the bioprosthetic valves herein are not limited thereto and can be used in other structures, including blood vessels.

In one embodiment, fresh biological tissue is first fixed or crossed-linked. Fixing or cross-linking can be accomplished by a number of techniques, for example, by cross-linking with epoxides, carbodimides or genipin, or photo fixation. Conventionally, fixing can be accomplished by cross-linking the amine groups of the tissue proteins with an aldehyde, such as a solution of about 0.001 v/v %-5 v/v % glutaraldehyde or formaldehyde for about several hours to several weeks. The fixed tissue preferably is rinsed thoroughly with a sterile saline solution to substantially reduce the amount of unreacted fixative within the tissue. Thereafter, the fixed tissue is further processed immediately or stored in an aqueous solution until further processing to prevent drying out and shrinkage of the fixed tissue.

In one embodiment, the aqueous storage solution may comprise about 0.001-10 v/v % of glutaraldehyde or formaldehyde. In another embodiment, the aqueous storage solution may be sterile saline. A saline solution is generally composed of distilled and/or deionized water and sodium chloride in a concentration ranging from about 0.01 wt % to about 1.5 wt %. More specifically, the sodium chloride concentration can range from about 0.75 wt % to about 1.05 wt %. Isotonic saline is often used. As an alternative to sodium chloride, the following salts may be used for the saline solution: potassium chloride, calcium chloride, magnesium sulfate, disodium phosphate, sodium bicarbonate, magnesium chloride, sodium phosphate, potassium phosphate, or any combination thereof, with or without sodium chloride. Additionally, the saline solution may be a balanced salt solution such as Hank's, Earle's, Gey's or Ouck's balanced salt solution, or may be a phosphate buffered solution. Treating the tissue with a saline solution may assist in leaching organic solvents or residue from the tissue.

After the fixing step (or after removal from the aqueous storage solution), the tissue is placed in an aqueous cellulose solution. The cellulose in the solution may include, but is not limited to, methyl cellulose, ethyl cellulose, hydroxypropyl cellulose and combinations thereof. Preferably, the aqueous cellulose solution comprises from about 1 wt % to about 50 wt % of cellulose; more preferably, from about 1 wt % to about 5 wt % of cellulose; and most preferably, about 2 wt % (viscosity 15 cps) of cellulose. The cellulose solution is non-toxic and is currently used in various industries (i.e., cosmetics, food, and medicine). Without wishing to be bound by any particular theory, cellulose is a large polymer that potentially could be trapped in the collagen matrix. Without an enzyme to cleave the linkages, the trapped cellulose will not degrade and could potentially reduce mechanical shear stress in the tissue and essentially function as a replacement for GAGs. Tissue treated with cellulose shows good mechanical stability and durability. The cellulose treatment may also help prevent change or damage to the tissue properties during the drying process. The cellulose remained on the surface of the tissue can be easily removed from the tissue by rinsing or rehydration, such as with a sterile saline solution.

The tissue component is contacted with the cellulose solution for a time and at a temperature sufficient to permit the cellulose solution to penetrate into the interstices of the tissue by passive diffusion and replace the fluid therein. The time needed to achieve such replacement is directly related to the thickness of the tissue, and inversely related to the ratio between the volume of the cellulose solution and the volume of the tissue. Tissue having a thickness between about 0.05 mm and about 2 mm may be contacted with a cellulose solution at a temperature between about 15° C. and about 30° C. for about one second to about one week; preferably, for at least 8 hours. More preferably, such tissue may be contacted with a cellulose solution at room temperature for about 12 to about 24 hours.

The tissue is contacted with the cellulose solution by a standard method, such as by immersion in the solution. The amount of the cellulose solution is at least sufficient to submerge the tissue. Preferably, the volume of the cellulose solution is at least about 2 times the volume of the tissue that is brought into contact with the solution; more preferably, about 50 times the volume of the tissue; and still more preferably, about 100 times the volume of the tissue.

After soaking in the cellulose solution for a sufficient time, the tissue is removed from the cellulose solution and optionally rinsed with a sterile saline solution. It then may be placed in an aqueous solution for storage. The aqueous storage solution is described above. In one embodiment, the cellulose-treated, fixed tissue can be stored in a package with an aqueous solution. The treated, fixed, wet tissue can be sterilized using an appropriate method that is compatible with the wet tissue Such method includes, but is not limited to, using various solutions, such as aldehydes, alcohols, epoxides or other various antimicrobial solutions, for liquid chemical sterilization.

Preferably, the cellulose-treated, fixed tissue is placed in a jar or other container for vacuum drying or in a lyophilizer for drying by lyophilization. Vacuum drying is a process in which materials are dried in a reduced pressure environment, which lowers the heat needed for rapid drying. As such, vacuum-drying tends to retain the integrity of the original item with less damage. A vacuum is applied to the container having the tissue inside for a time sufficient to remove substantially all the fluid in the tissue, without the application of heat. The vacuum pressure is no more than about 1000 mTorr, preferably no more than about 200 mTorr, and more preferably no more than about 10 mTorr. The tissue can be subjected to vacuum drying for about one minute to about 2 months. Preferably, a vacuum of about 5 mTorr is applied to the container for at least 4 hours. More preferably, a vacuum of about 5 mTorr is applied to the container for about 6 to about 24 hours.

In some instances, the tissue resulting from the cellulose solution treatment is dried by freezing drying, also called lyophilization. Lyophilization techniques generally involve freezing the tissue, followed by drying the frozen tissue by a sublimation process. Some representative lyophilization conditions that may be used in methods for making lyophilized tissue for use in a prosthetic heart valve include: 1) freezing the tissue at about −20° C. for at least 12 hours, followed by a single vacuum-drying step at, for example, 150 mTorr at room temperature for 15-20 hours; 2) freezing at about −70° C. for at least two hours, followed by annealing (subjecting the frozen tissue to an increased temperature, such as about −20° C., for at least one hour), then again reducing the temperature to about −70° C. for at least two more hours, followed by drying at a reduced pressure of about 150 mTorr at room temperature for at least 12 hours; 3) freezing at about −40° C. for at least two hours, followed by annealing as discussed previously, again reducing the temperature to about −40° C. for at least two more hours, and then two-stage drying—a first stage at about −5° C. and about 160 mTorr for at least two hours, followed by a second stage at room temperature and about 160 mTorr for at least two hours; and 4) freezing in liquid nitrogen (at about −210° C. to about −196° C.) for at least two hours and up to about two months, followed by annealing as discussed previously. Various combinations of these and other parameters may also be used, or one or more of the processes may be used in series. The dried tissue is stored in a dry, ambient environment.

In a preferred embodiment, prior to the fixing step, the fresh biological tissue is placed in an aqueous cellulose solution. This preliminary step of cellulose treatment is the same as the cellulose treatment step described above. After the preliminary cellulose treatment step, the biological tissue optionally may be rinsed and then subjected to the steps described above, including fixing and treating with a second aqueous cellulose solution. The resultant tissue can be stored in an aqueous solution. Preferably, the resultant tissue is further subjected to vacuum drying or lyophilization, and stored in a dry, ambient environment. In a variant of this last method, when the biological tissue is treated with an aqueous cellulose solution prior to fixing, the second cellulose treatment step (after fixing) can be omitted, regardless of whether or not the resultant tissue is dried by vacuum or lyophilization.

In another embodiment, the step of treating the tissue with a cellulose solution may be performed during the step of fixing the fresh biological tissue. In one embodiment, the fresh biological tissue is treated with a solution comprising cellulose and a cross-linking chemical reagent such as epoxides, carbodimides, genipin or aldehydes. The solution may contain about 0.001-5 v/v % glutaraldehyde or formaldehyde as a cross-linking reagent. The cellulose in the solution may include, but is not limited to, methyl cellulose, ethyl cellulose, hydroxypropyl cellulose and combinations thereof. Preferably, the solution comprises between about 1 wt % and about 50 wt % of cellulose; more preferably, between about 1 wt % and about 5 wt % of cellulose; and most preferably, about 2 wt % (viscosity 15 cps) of cellulose. The amount of the solution is at least sufficient to submerge the tissue. Preferably, the volume of the solution is at least about 2 times the volume of the tissue that is brought into the contact with the solution; more preferably, about 50 times the volume of the tissue; and still more preferably, about 100 times the volume of the tissue. The tissue component is contacted with this solution for a time and at a temperature sufficient to cross-link the fresh biological tissue and at the same time permit cellulose to penetrate into the interstices of the tissue by passive diffusion and replace the fluid therein. Tissue having a thickness of between about 0.05 mm and about 2 mm may be contacted with such a solution at a temperature between about 15° C. and about 30° C. for about one second to about one week; preferably, for at least 8 hours. More preferably, such tissue may be contacted with such a solution at room temperature for about 12 to 24 hours.

The cellulose treatment of the tissue and the drying steps described above may be performed on individual tissue components for the valve assembly, on a valve assembly, or on a completed tissue valve. Thus, individual tissue components may be subjected to the cellulose treatment step prior to being cross-linked and formed into a valve assembly, after formation of the valve assembly but prior to the fabrication of the tissue valve, or after fabrication of the tissue valve.

Vacuum-drying or lyophilization of the tissue can occur before or after construction of a valve assembly or before or after construction of a tissue valve. Thus, in one embodiment, the cellulose-treated, “wet” fixed tissue (bioprosthetic fixed tissue treated with a cellulose solution) is vacuum dried or lyophilized and then stored dry until needed for manufacture. The tissue may need to be rehydrated prior to being manufactured into a valve assembly, and may then be vacuum dried or lyophilized again for storage. In another embodiment, the cellulose-treated, “wet” fixed tissue is attached to a support or stent to construct a valve assembly on the support and thereafter the entire resulting tissue valve is vacuum dried or lyophilized prior to storage. In yet another embodiment, the cellulose-treated, “wet” fixed tissue is used to construct a “wet” valve assembly without a support. This “wet” valve assembly can be stored in an aqueous solution. It can also be vacuum dried or lyophilized, and then attached to a support; or attached to the support first, followed by vacuum-drying or lyophilization of the resulting tissue valve.

The construction of a valve assembly or a tissue valve can involve the general techniques used in the art. An example of a method of constructing a tissue valve includes attaching a valve assembly comprised of at least one leaflet to a support or stent configured to fit within the relevant structure within the patient. The support could either have a fixed size in the case of a surgical valve, or could be collapsible in the case of a transcatheter valve implanted using a minimally invasive procedure.

Due to the removal of fluid from the bioprosthetic tissue during the vacuum-drying or lyophilization process, the resulting “dry” bioprosthetic valve has a reduced size, volume and weight when compared to a tissue valve that was stored in a fluid medium. Normally, the weight of “dry” bioprosthetic tissue is at least about 50%, preferably about 75-80%, less than the weight of the same tissue in the wet state.

The method of producing tissue valves for dry storage in the present application eliminates the use of toxic fluids associated with the storage of valves and the subsequent rinsing process, which may make the manufacture and transport of the valves less expensive, may make storage and use more convenient, and may result in less chemical waste to dispose of. It also may reduce the time spent in the operating room preparing the tissue valve for implantation.

Since the tissue valves prepared by the methods described herein do not need to be shipped or stored in a solution to prevent the tissue from drying out, they may be pre-loaded onto delivery devices, with the entire assembly being provided in sterile packaging such that the valves are able to be reconstituted before use.

The dry tissue component, valve assembly or bioprosthetic valve comprising the dry tissue component, wherein the tissue has been treated with cellulose prior to, during, after, or both prior to and after fixing, is stored in an environment essentially free of liquid for later processing or implantation. An environment, container or package that is “essentially free of liquid” as used herein means an environment in which the presence of water or other liquids is limited to the content of such liquids in the ambient air (as more precisely expressed as the relative humidity), and the content of liquid contained within the treated tissue disposed within the container or package. Preferably, the treated dry tissue component, valve assembly or bioprosthetic valve made therefrom is placed into a microorganism-resistant package. An example of a packaged tissue valve ready for reconstitution includes a package having an outer periphery that defines an inner space. The inner space has an environment that is substantially dry and sterile. The tissue valve includes a support configured to fit within the inner space of the package and a vacuum dried or lyophilized valve assembly attached to the support. The dry tissue valve is encased within the package. In one embodiment, the package with the dry tissue valve may also include a delivery device. In another embodiment, the dry tissue valve may be preloaded within the delivery device within the package.

After the dry tissue component, valve assembly or bioprosthetic valve has been placed in the inner space of the package, the package is sealed. The sealed package may then be sterilized, such as by a gas sterilization process or by exposure to ionizing radiation. An example of a conventional procedure for sterilization by exposure to ethylene oxide involves exposure of the package to a mixture of 10 wt % ethylene oxide and 90 wt % hydrochlorofluorocarbon at a chamber pressure of about 8 to 10 psi and a temperature of about 38° C. for about 24 hours, or a temperature of about 54-57° C. for about 130 minutes. To ensure the inner space remains sterile following sterilization, the package is preferably formed from a material that is impenetrable to microorganisms such as bacteria and fungi.

The resulting product is a substantially sterile tissue component, valve assembly or implantable tissue or bioprosthetic valve suitable for dry storage. The sterile bioprosthetic tissue valve prepared in accordance with the present methods may be well-suited for implantation into patients with cardiovascular diseases. As used herein, the term “patient” means any mammals such as, for example, humans, dogs, cats, horses, and non-human primates. Prior to use, the bioprosthetic valve is removed from the package, and the tissue portion thereof is rehydrated by exposure to an aqueous solution, preferably a sterile saline solution, such as a sterile 0.9 wt % saline solution. In some instances, the tissue portion is rehydrated by rinsing with a sterile 0.9 wt % saline solution for about two to about ten seconds, and then loaded into or onto a delivery device. When a substantially sterile tissue component or valve assembly is stored in the package, the tissue component or valve assembly may be rehydrated using the same technique as for the bioprosthetic valve, after which the tissue component or valve assembly is assembled to a support to form a bioprosthetic valve.

Rehydration or reconstitution of the vacuum-dried or lyophilized tissue before use may be a complete reconstitution in which the moisture content of the tissue is roughly equivalent to the moisture content the tissue would have when in equilibrium with the patient's biofluid in situ. In such circumstances, and in some embodiments, the delivery device may be configured to accommodate a resulting increase in the volume of the rehydrated bioprosthetic valve, if needed.

In one embodiment, the tissue valve comprises a vacuum-dried or lyophilized valve assembly attached to a stent that is capable of being resheathed, with the resulting tissue valve loaded onto a delivery device which permits resheathing. The stent and the valve assembly are configured cooperatively such that the valve assembly can be exposed when on the delivery device, reconstituted, sheathed/resheathed and/or implanted. This allows the surgical team to expose the valve assembly for reconstitution prior to implantation. In a further aspect of this embodiment, the stent may be structured such that the valve assembly can be exposed for reconstitution. In a further embodiment, a bioprosthetic valve which is not preloaded onto a delivery device may be reconstituted and thereafter crimped and loaded onto a delivery device.

In another embodiment, the valve assembly may be reconstituted while fully sheathed. Reconstitution while fully sheathed may be achieved by irrigating an aqueous solution through the delivery device prior to implantation.

The presently described methods of producing bioprosthetic tissue valves for dry storage, which comprise treating the tissue with a cellulose solution and subsequently vacuum-drying or freeze-drying by lyophilization, reduce changes or damage to the tissue and the valve made therefrom during the drying process. As a result, tissue treated in accordance with the present methods can be returned to a size that is at least about 90%, preferably at least about 95%, more preferably at least about 98%, of its original hydrated size following rehydration in sterile saline for about 10 seconds. The tissue prepared in accordance with the present methods is therefore well-suited for use in a bioprosthetic valve.

The following examples are for purpose of illustration only and are not intended to limit the scope of the invention as defined in the claims hereinafter.

### Example 1

One liter of a 2 wt % methyl cellulose solution was prepared by heating 300 ml of water to at least 80° C., and adding 20 g of methyl cellulose powder to the hot water with agitation. The mixture was further agitated until the methyl cellulose particles were thoroughly wetted and evenly dispersed. 700 ml of cold water or ice was then added to lower the temperature of the dispersion. Once the dispersion cooled to a temperature at which the methyl cellulose became water soluble, the powder began to hydrate and the viscosity of the solution increased. The resulting solution was then cooled in a bath at 0-5° C. for about 20 to 40 minutes under continuous agitation. The solution was continually agitated for at least 30 minutes after room temperature was reached. The viscosity of the resulting methyl cellulose solution was about 15 cPs.

Two 25 mm Portico® aortic valves (cryocut) were stored in jars containing 0.5% glutaraldehyde storage solution. The storage solution was decanted and then replaced with a sufficient amount of 2 wt % methyl cellulose solution to cover the valves.

The valves were kept in the methyl cellulose solution for about 26 hours at room temperature. The methyl cellulose solution was then decanted from the valve jars. The valves were then dried by applying a vacuum of 200 mTorr to the valve jars (with lids removed and the valves inside) for about 26 hours at room temperature. The valve jars were then removed from the vacuum appliance and the lids were placed on the jars.

Some residual methyl cellulose was observed in the valve jars and on the dried valves. After a rinse with 0.9 wt % saline for about 10 seconds twice, the tissue leaflets were pliable. The rinse appeared to remove most of the residual methyl cellulose and rehydrated the valves.

Each rehydrated aortic valve was assembled in a pneumatic-actuated left-sided heart model having a mechanical mitral valve, and tested under pulsatile flow. Controlling and tuning of the system was performed using a custom National Instrument (Austin, Tex.) LabView® acquisition system to measure flow and pressure at the inflow and outflow of the aortic valve over 10 cardiac cycles. Parameters measured at 70 pulses per minute, 100 mm Hg aortic mean pressure, and 5 L/minute flow included effective orifice area (EOA), peak pressure drop, and regurgitation.

The baseline hydrodynamic test results are shown in Table 1.

TABLE 1

| Parameter | Valve #1 | Valve #2 |
|---|---|---|
| EOA (cm²) | 2.73 | 2.66 |
| Peak Pressure Drop (mmHg) | 7.4 | 8.5 |
| % Regurgitation | 10.3 | 9.0 |

### Example 2

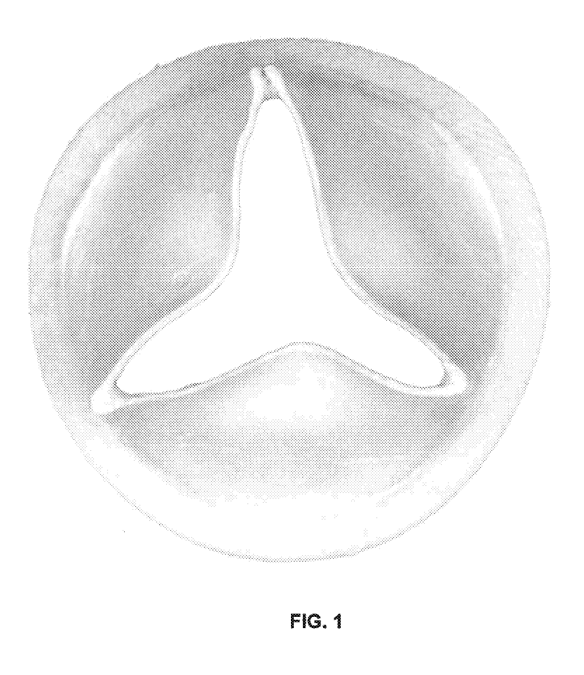

One 25 mm Trifecta® aortic valve was prepared according to the process in Example 1. An image of the dry valve is shown in FIG. 1.

### Example 3

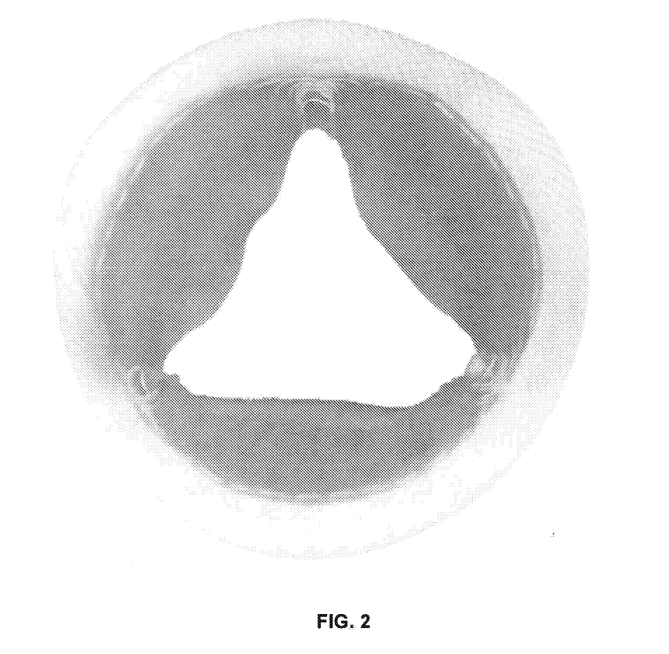

One 25 mm Trifecta® aortic valve was prepared according to the process in Example 1, except the valve was air dried for about 26 hours, rather than vacuum dried. An image of the dry valve is shown in FIG. 2.

The vacuum-dried valve prepared in Example 2 was flexible. After being rehydrated, it retained its form with little change in tissue dimensions. In contrast, the air-dried valve prepared in Example 3 significantly contracted and became very stiff. It did not retain its pre-dried properties after being rehydrated.

To summarize the foregoing description, a method of processing biological tissue may include providing fresh biological tissue; treating the tissue with an aqueous cellulose solution for a time sufficient to allow fluid in the tissue to be replaced by the aqueous cellulose solution; and cross-linking the tissue, wherein the treating step is performed prior to, during, after, or both prior to and after the cross-linking step; and/or the method may further comprise storing the cellulose-treated cross-linked tissue in an aqueous solution; and/or

the method may further comprise placing the cellulose-treated cross-linked tissue in a package with an aqueous solution; and/or

the method may further comprise drying the cellulose-treated cross-linked tissue by a vacuum-drying or lyophilization process to form dried tissue; and storing the dried tissue in a dry, ambient environment; and/or

the vacuum-drying process may use a vacuum of no more than 1000 mTorr; and/or

the vacuum-drying process may use a vacuum of no more than 200 mTorr; and/or

the drying step may be performed for at least 4 hours; and/or
the method may further comprise placing the dried tissue in a package and sealing the package; and/or

the method may further comprise sterilizing the package after the sealing step; and/or

the cross-linking step may be performed chemically; and/or

the aqueous cellulose solution may include a cross-linking chemical reagent; and/or

the cross-linking chemical reagent is one or more selected from the group consisting of epoxides, carbodimides or genipin, and aldehydes; and/or

the aqueous cellulose solution may comprise cellulose selected from the group consisting of methyl cellulose, ethyl cellulose, hydroxypropyl cellulose and combinations thereof; and/or

the cellulose may be methyl cellulose; and/or

the aqueous cellulose solution may comprise between about 1% and about 50% by weight of cellulose; and/or

the aqueous cellulose solution may comprise about 2% by weight of cellulose; and/or

the aqueous cellulose solution may have a volume sufficient to submerge the tissue; and/or

the treating step may be performed for about one second to about one week; and/or

the treating step may be performed for at least 8 hours; and/or
the treating step may be performed at a temperature between about 15° C. and about 30° C.; and/or

the tissue may be in the form of a tissue component, a valve assembly for a bioprosthetic valve or a fully assembled bioprosthetic valve incorporating the tissue; and/or

the prosthetic valve may be a prosthetic heart valve; and/or
the prosthetic valve may include a stent.

The present application also includes the tissue prepared by the described methods, and a valve assembly for a bioprosthetic valve or a fully assembled bioprosthetic valve incorporating the tissue.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of processing tissue, comprising:
providing fresh biological tissue;
treating the tissue with an aqueous cellulose solution for a time sufficient to allow fluid in the tissue to be replaced by the aqueous cellulose solution; and
cross-linking the tissue,
wherein the treating step is performed prior to, during, after, or both prior to and after the cross-linking step.

2. The method of claim 1, further comprising storing the cellulose-treated cross-linked tissue in an aqueous solution.

3. (canceled)

4. The method of claim 1, further comprising
drying the cellulose-treated cross-linked tissue by a vacuum-drying or lyophilization process to form dried tissue; and
storing the dried tissue in a dry, ambient environment.

5. (canceled)

6. The method of claim 4, wherein the vacuum-drying process uses a vacuum of no more than 200 mTorr.

7. The method of claim 4, wherein the drying step is performed for at least 4 hours.

8. The method of claim 4, further comprising placing the dried tissue in a package and sealing the package.

9. The method of claim 8, further comprising sterilizing the package after the sealing step.

10. The method of claim 1, wherein the cross-linking step is performed chemically.

11. The method of claim 1, wherein the aqueous cellulose solution includes a cross-linking chemical reagent.

12. The method of claim 11, wherein the cross-linking chemical reagent is one or more selected from the group consisting of epoxides, carbodimides or genipin, and aldehydes.

13. The method of claim 1, wherein the aqueous cellulose solution comprises cellulose selected from the group consisting of methyl cellulose, ethyl cellulose, hydroxypropyl cellulose and combinations thereof.

14. (canceled)

15. The method of claim 1, wherein the aqueous cellulose solution comprises between about 1% and about 50% by weight of cellulose.

16. The method of claim 1, wherein the aqueous cellulose solution comprises about 2% by weight of cellulose.

17. The method of claim 1, wherein the aqueous cellulose solution has a volume sufficient to submerge the tissue.

18. The method of claim 1, wherein the treating step is performed for about one second to about one week.

19. (canceled)

20. The method of claim 1, wherein the treating step is performed at a temperature between about 15° C. and about 30° C.

21. The method of claim 1, wherein the tissue is in the form of a tissue component, a valve assembly for a bioprosthetic valve or a fully assembled bioprosthetic valve incorporating the tissue.

22. The method of claim 21, wherein the prosthetic valve is a prosthetic heart valve.

23. (canceled)

24. (canceled)

25. Tissue prepared according to the method of claim 1.

26. Tissue prepared according to the method of claim 4.
